(19) United States
(12) Patent Application Publication
(10) Pub. No.: US 2025/0040942 A1
(43) Pub. Date: Feb. 6, 2025

(54) STATISTICAL METHODS AND SYSTEMS FOR DETECTING PERFORATIONS DURING SURGICAL DRILLING BASED ON SENSED ELECTRICAL CHARACTERISTICS

(71) Applicants: SpineGuard, Vincennes (FR); Sorbonne Universite, Paris (FR); Centre National De La Recherche Scientifique-CNRS, Paris (FR); INSERM (Institut National de la Sante et de la Recherche Medicale), Paris (FR)

(72) Inventors: Lilyan LEBLANC, Paris (FR); Brahim TAMADAZTE, Paris (FR); Thibault CHANDANSON, Vincennes (FR); Elie SAGHBINY, Paris (FR)

(73) Assignees: SpineGuard, Vincennes (FR); Sorbonne Universite, Paris (FR); Centre National De La Recherche Scientifique-CNRS, Paris (FR); INSERM (Institut National de la Sante et de la Recherche Medicale), Paris (FR)

(21) Appl. No.: 18/784,828

(22) Filed: Jul. 25, 2024

Related U.S. Application Data
(60) Provisional application No. 63/516,490, filed on Jul. 28, 2023.

(30) Foreign Application Priority Data
Jul. 27, 2023 (EP) 23306301.5

(51) Int. Cl. A61B 17/17; A61B 17/00; A61B 17/16; A61B 17/56; A61B 34/30; A61B 90/00; G16H 20/40; G16H 40/63

(52) U.S. Cl. CPC A61B 17/1707; A61B 17/1671; A61B 17/1757; A61B 34/30; G16H 20/40; G16H 40/63; A61B 2017/00026; A61B 2017/564; A61B 2090/08021

(57) ABSTRACT

A medical device for penetrating an anatomic structure, e.g., a bone structure, including a processing unit programmed to execute one or more statistical algorithms, e.g., Bayesian-based perforation detection algorithms, with electrical conductivity measured during penetration of an anatomic structure as an input to detect a breach condition, e.g., a spinal canal perforation, based on the measured electrical conductivity. The medical device may include a drill bit having sensing capabilities coupled to the distal end of a robot arm via a power drill unit mounted on the robot arm. The power drill unit may cease transmission of rotary motion to the drill bit upon detection of the breach condition.

### CROSS-REFERENCE TO RELATED APPLICATION

This application claims priority to U.S. Prov. Patent Appl. No. 63/516,490, filed Jul. 28, 2023, and to European Patent Appl. No. 23306301.5, filed Jul. 27, 2023, the entire contents of each of which are incorporated herein by reference.

### FIELD OF USE

The present disclosure relates to a medical device for penetrating an anatomical structure, a medical system comprising such a medical device, and methods of use.

### BACKGROUND

The principles of the present invention apply to any type of surgical intervention on an anatomical structure made up of anatomical media having different electrical conductivities.

The invention nonetheless applies very particularly in orthopedic surgery and in surgery of the spine, in which one or more penetrating medical devices are used by a surgeon to penetrate an anatomical structure comprising a bone structure and, in particular, to drill bone structure, for example to position or attach a prosthesis or an implant.

Pedicle screw (PS) placement in spine surgery has been considered a standard surgical procedure for the past forty years. It is widely used for various spinal procedures, especially in the case of spinal deformities, e.g., scoliosis. Scoliosis treatment depends on the angular deformity's etiology and severity. Surgical intervention is usually indicated for more severe deformities, e.g., those greater than 50° angular deformation. The surgical treatment, also known as arthrodesis, consists of spinal fusion by implantation of PS and rods. The PS should be positioned correctly to obtain maximum stability of the bone-screw interface while avoiding a perforation in the spinal canal during insertion. Drilling the pedicles for screw positioning is a challenging procedure as failure can lead to injuries to major vessels (aorta, vena cava) or the spinal cord. The literature reports a complication rate in the case of screw malpositioning of up to 18%.

Among the bone structures on which the surgeon may operate, some include an outer layer of cortical bone enclosing a layer of trabecular bone, the layer of trabecular bone at least partially covering a layer of internal cortical bone. The anatomical structure also may include soft tissues bordered by the layer of internal cortical bone which then forms an interface between the layer of trabecular bone and the soft tissues. During a procedure, it is important to prevent damage to functional tissues, such as tissues of the nervous or vascular system, located near the outer cortical bone layer or in soft tissues. This is particularly the case for an intervention on a vertebral pedicle in which the nerve roots are close to the outer cortical bone layer and the spinal cord constitutes part of the soft tissues bordered by the internal cortical bone layer forming the vertebral foramen.

Detecting a potential breach when drilling a bone based on various measurements including force, speed, position, and conductivity, e.g., detecting an unexpected change in the measured signal, has consequently become an active research field. For example, an algorithm for stapedotomy based on detecting the simultaneous increase of torque and the decrease of the feed force during the drilling procedure has been proposed. See, e.g., P. N. Brett, D. A. Baker, Reyes, et al., “An Automatic Technique for Micro-Drilling a Stapedotomy in the Flexible Stapes Footplate,” J. of Eng. in Med., vol. 209, no. 4, pp. 255-262, 1995. Similarly, it has been suggested to track the gradient of the force signal (after filtering) to detect unexpected variations in the signal, indicating that the drill is breaching a femur bone [27]. See, e.g., B. Allotta, F. Belmonte, Bosio, et al., “Study on a mechatronic tool for drilling in the osteosynthesis of long bones: Tool/bone interaction, modeling and experiments,” Mech., vol. 6, pp. 447-459, 1996. In addition, it has been proposed to use the trend and a threshold on both the torque and feed rate on a variety of bone types (e.g., scapula, skulls, and femurs). See, e.g., W.-Y. Lee and C.-L. Shih, “Control and breakthrough detection of a three-axis robotic bone drilling system,” Mechatronics, vol. 16, no. 2, pp. 73-84, 2006. Although these methods are promising, qualitative and quantitative evaluations are not provided.

It has further been suggested to use feature functions based on the measured force for different drill shapes. See, e.g., Y. Hu, H. Jin, L. Zhang, et al., “State Recognition of Pedicle Drilling With Force Sensing in a Robotic Spinal Surgical System,” IEEE/ASME T. on Mech., vol. 19, no. 1, pp. 357-365, 2014. In this study, cattle vertebrae drilling signals were used to identify threshold values which were then used to stop the drilling when the force reaches the transitional zone from the cancellous tissue into the cortical tissue. In addition, monitoring electrical current through a drill motor has been used to detect unexpected changes in the signal to identify a potential breach within the safety zone. See, e.g., Y.-L. Hsu, S.-T. Lec, and H.-W. Lin, “A modular mechatronic system for automatic bone drilling,” Biomed. Eng.: Appl., Basis and Comm., vol. 13, no. 04, pp. 168-174, 2001. However, no metric evaluation was reported on sample size, success rate, or detection accuracy.

To assist the surgeon in preventing damage to functional tissues, it is known to obtain information on positioning of the penetrating medical device, e.g., drill bit, with respect to the different anatomical media of the anatomical structure based on their respective electrical conductivities, which are representative of the ability of the media to conduct an electric current. For example, the layer of trabecular bone and the soft tissues constitute first and second anatomical media respectively having first and second electrical conductivities, the first electrical conductivity being lower than the second electrical conductivity. The cortical bone layer constitutes a third anatomical medium and has a third electrical conductivity, lower than the first and second electrical conductivities.

Hand tools are known in medical applications for exploiting differences in electrical conductivity of the media comprising a bone structure. For example, the manually manipulated medical device marketed under the name of PediGuard® (made available by SpineGuard SA, Paris, France), described in document WO 03/068076, uses such differences as measured by Dynamic Surgical Guidance (DSG) sensors to vary a warning signal perceptible by the surgeon, so to alert the surgeon when damage to functional tissue is occurring or is imminent. This method has shown 100% of perforation detection over 104 performed drillings in a +2 mm range, with an average accuracy of 0.65 mm after the perforation (STD=0.4 mm). See, e.g., J. Da Silva, E. Saghbini, T. Chandanson, et al., “Automatic bone breach detection for spine surgery based on bio-electrical conductivity sensing: Ex-vivo experimental validation,” in Conf. on New Tech. for Comp. and Rob. Ass. Surg., 2022. The differences in electrical conductivity of the media of the anatomical structure comprising a bone structure have also been exploited in medical applications implementing an at least partially automated medical system subject in part to automatic control, and in particular, in robotics. For example, the medical system described in WO 2019/081850, uses these differences to vary a warning signal used in a control signal controlling movement of a robotic arm and to modify the control signal when the warning signal indicates that damage to functional tissue is occurring or imminent.

Although navigation and robotic approaches have contributed to improving spine surgical procedures, they present several limitations, particularly in terms of usability and accuracy. Accuracy is reduced because of the patient's breathing movement. Furthermore, a subtle change in the patient's position during the pedicle preparation is sufficient to introduce registration errors.

Although these foregoing systems are beneficial, it would be desirable to provide systems and methods that offer improved precision and more reliable discrimination of anatomical media during the penetration into an anatomical structure comprising a bone structure, thereby to better ensure patient safety. Such improved systems and methods will find applicability in any intervention on any type of anatomical structure, whether or not it requires penetrating a bone structure.

In view of the foregoing drawbacks of previously known systems and methods, there exists a need for a suitable and effective perforation detection algorithm capable of identifying meaningful changes in the trend of signals of interest, operating in real-time, robust to measurement noise (regardless of the sensor type), and having minimal parameter tuning. Accordingly, the surgeon should not need to perform complex parameterization before or during the surgical procedure, e.g., depending on the vertebral bone quality.

### SUMMARY

The present disclosure overcomes the drawbacks of previously-known systems and methods by providing a method implemented by a system comprising one or more processors for penetrating an anatomic structure. The method may include: causing a drilling portion to penetrate the anatomic structure; receiving data indicative of electrical conductivity sensed by the drilling portion as the drilling portion penetrates the anatomic structure; using a probabilistic perforation detection algorithm to probabilistically detect a breach condition based on the data; and causing, if the breach condition is detected, the drilling portion to stop or modify penetration of the anatomic structure. Using the probabilistic perforation detection algorithm to probabilistically detect the breach condition based on the data may include using the Bayesian-based perforation detection algorithm to probabilistically detect a time instant when a probability distribution of a time series changes. Moreover, using the Bayesian-based perforation detection algorithm to probabilistically detect the time instant when the probability distribution of the time series changes may include using the Bayesian-based perforation detection algorithm to probabilistically detect the time instant when a run length associated with the data drops to zero.

The probabilistic perforation detection algorithm may be a Bayesian-based perforation detection algorithm, such that using the Bayesian-based perforation detection algorithm to probabilistically detect the breach condition based on the data may include: calculating a posterior predictive based on the data; calculating a growth probability based on the posterior predictive; calculating a changepoint probability; calculating a marginal probability; calculating a run length distribution based on the changepoint probability and the marginal probability; computing a run length based on the run length distribution; determining whether the run length exceeds a detection threshold once; and probabilistically detecting the breach condition if the run length falls below the detection threshold after exceeding the detection threshold once.

In addition, using the Bayesian-based perforation detection algorithm to probabilistically detect the breach condition based on the data may include assuming a changepoint prior is constant. Moreover, using the Bayesian-based perforation detection algorithm to probabilistically detect the breach condition based on the data may include assuming a normal likelihood with an unknown mean and variance for the data. Calculating the posterior predictive based on the data may include using a conjugate exponential model to allow sequentially updating one or more distribution parameters as the data is received. Moreover, using the conjugate exponential model may include using a conjugate prior such that a posterior is in a same distribution as a prior. The conjugate prior may be a Normal-Inverse-Gamma distribution, and the run length distribution may be a generalized Student's T distribution based on one or more distribution parameters. In addition, the method further may include initializing the one or more distribution parameters with one or more pro-perforation distribution priors, such that calculating the posterior predictive may include using one or more post-perforation distribution priors.

In addition, probabilistically detecting the breach condition if the run length falls below the detection threshold after exceeding the detection threshold once may include probabilistically detecting the breach condition if the run length falls below the detection threshold in a predetermined [0, N] range. The method further may include modeling growth of an existing run associated with the data by calculating the posterior predictive using one or more distribution parameters associated with the data received at a first time for each run length value before updating each run length parameter value based on the data received at a second time. In some embodiments, using the Bayesian-based perforation detection algorithm to probabilistically detect the breach condition based on the data may not require filtering of the data or prior calibration.

The method further may include: determining at least one of an entry point into the anatomic portion or a trajectory along which the drilling portion penetrates the anatomic structure; and causing a robot arm coupled to the drilling portion to position the drilling portion in alignment with the at least one of the entry point or the trajectory. For example, the robot arm may be coupled to the drilling portion via a power drill unit mounted on a distal end of the robot arm, the power drill unit configured to transmit rotary motion to the drilling portion. In addition, causing the drilling portion to penetrate the anatomic structure may include causing a power drill unit coupled to the drilling portion to transmit rotary motion to the drilling portion.

In accordance with another aspect of the present disclosure, a system for penetrating an anatomic structure is provided. The system may include a drilling portion configured to sense electrical conductivity as the drilling portion penetrates the anatomic structure, and a controller operatively coupled to the drilling portion. The controller may be programmed to: cause the drilling portion to penetrate the anatomic structure; receive data indicative of electrical conductivity sensed by the drilling portion as the drilling portion penetrates the anatomic structure; use a probabilistic perforation detection algorithm to probabilistically detect a breach condition based on the data; and cause, if the breach condition is detected, the drilling portion to stop or modify penetration of the anatomic structure. The system further may include a robot arm and a power drill unit mounted on the robot arm. The power drill unit may be operatively coupled to the controller and may be configured to transmit rotary motion to the drilling portion to penetrate the anatomic structure.

### BRIEF DESCRIPTION OF THE DRAWINGS

FIG. 1 illustrates an exemplary surgical drilling system constructed in accordance with the principles of the present disclosure.

FIG. 2A illustrates an exemplary surgical drill bit for use with the surgical drill system of FIG. 1.

FIG. 2B illustrates an alternative exemplary surgical drill bit constructed in accordance with the principles of the present disclosure.

FIG. 2C illustrates an exemplary surgical burr with sensing capabilities constructed in accordance with the principles of the present disclosure.

FIG. 3 is a schematic diagram of an exemplary controller constructed in accordance with the principles of the present disclosure.

FIG. 4A illustrates a lateral foraminal breach and an anterolateral breach during pedicle screw placement, FIG. 4B illustrates an exemplary spinal canal perforation during pedicle screw placement, and FIG. 4C illustrates a well-placed pedicle screw.

FIG. 4D illustrates exemplary electrical conductivity along a drilling trajectory leading to the perforation of FIG. 4A.

FIG. 4E illustrates exemplary electrical conductivity along a drilling trajectory leading to the perforation of FIG. 4B.

FIG. 5 illustrates exemplary conductivity signals acquired during a vertebra drilling process over time.

FIG. 6A illustrates exemplary univariate data samples observed during a vertebra drilling process over time.

FIG. 6B illustrates exemplary run lengths observed during a vertebra drilling process over time.

FIG. 6C illustrates an exemplary recursive message-passing algorithm for the joint distribution over the current run length and the data observed at a given time instant.

FIG. 7 illustrates examples of electrical conductivity signals for which perforation was detected during a vertebra drilling process.

FIG. 8A illustrates the distance of an automatic stop to a perforation point for various drilled vertebrae in accordance with the principles of the present disclosure.

FIG. 8B is the histogram of all the computed distances for the drilled vertebrae of FIG. 8A.

FIG. 9 illustrates a classification of unsupervised methods for changepoint detection.

### DETAILED DESCRIPTION

Surgical drilling systems and methods are provided for performing surgical procedures, such as surgical drilling into bony structures, while guided by a conductivity sensing system. Systems configured in accordance with the principles of the present disclosure include a drill bit having conductivity sensing capabilities coupled to a robotic arm of a surgical robotic system via a power drill unit configured to provide power to and actuate the drill bit. The systems further include a controller programmed to execute one or more algorithms, e.g., statistical perforation detection algorithms, to detect one or more breach conditions during penetration of an anatomic portion by the drill bit, such that the power drill unit automatically arrests advancement of the drill bit responsive to the sensed conductivity by the drill bit in near real-time, e.g. within a few milliseconds to seconds, to thereby prevent injury to the patient. The statistical perforation detection algorithms may be stored and executed by conductivity sensing systems such as those described in U.S. Patent Appl. Publ. Nos. 2022/0361896 to Bette and 2023/0095197 to Chandanson, the entire contents of each of which are incorporated herein by reference.

Systems described herein are particularly advantageous for use in the field of orthopedic surgery and spine surgery to assist a surgeon during a surgical procedure in placing an implant in one or more vertebrae of a patient's spine. The robot arm enables improved placement precision of the drill bit relative to the patient and the conductivity sensing system prevents risk of damage related to unintended intrusion into sensitive functional tissues, such as the spinal cord, nerve endings, and vascular structures. Although the devices and methods of the present disclosure are described herein with respect to an application in a vertebra, and more generally in bony structure, they are not limited to such an application. Instead, the principles of the present disclosure advantageously may be applied to any anatomic portion comprising different mediums and having an electrical characteristic, such as a conductivity or resistivity, which varies as a function of the capacities of the mediums to conduct an electric current.

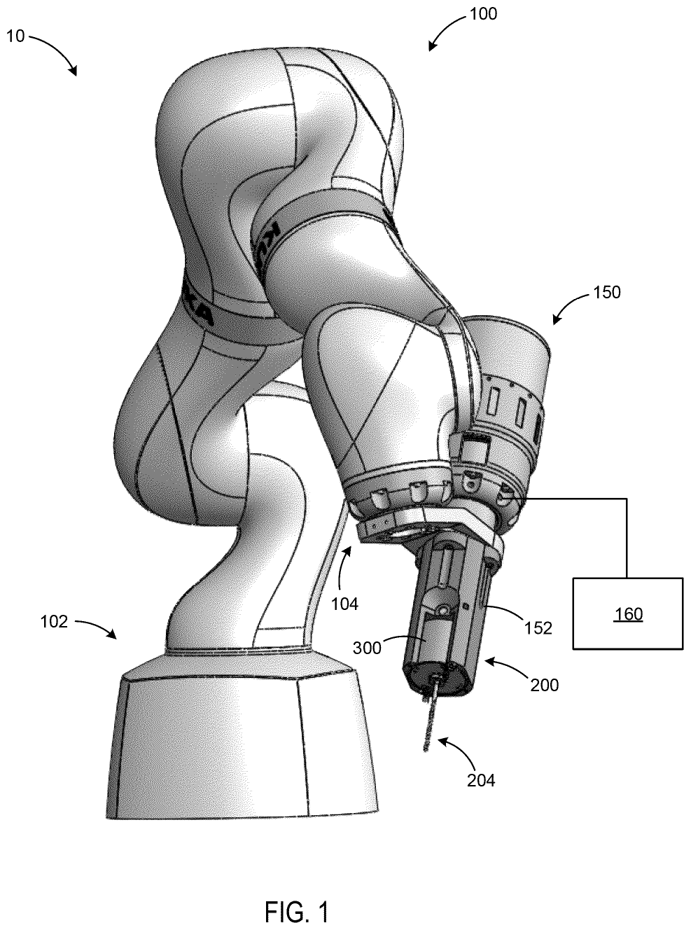

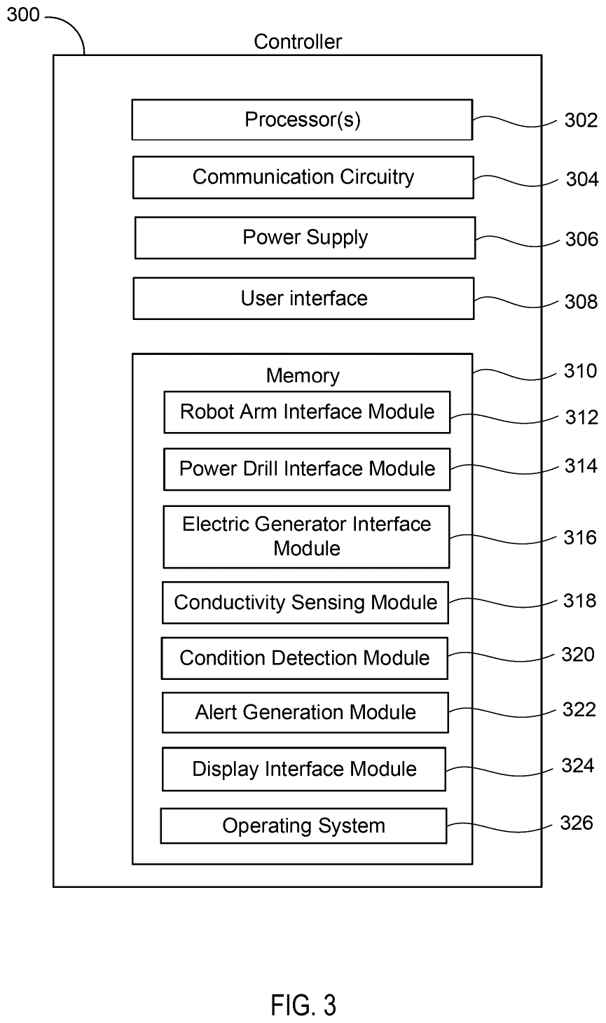

Referring now to FIG. 1, an exemplary surgical drilling system configured in accordance with one aspect of the present disclosure is described. As shown in FIG. 1, surgical drilling system 10 includes robot arm 100, power drill unit 150, drill bit 200, and controller 300, e.g., an electric processing device mounted on a circuit board. Controller 300 may be disposed entirely within robot arm 100, power drill unit 150, and/or drill bit 200, e.g., a proximal housing of drill bit 200, or a combination of robot arm 100, power drill unit 150, and/or drill bit 200, or external to robot arm 100, power drill unit 150, and drill bit 200, e.g., in external computing device 160 operatively coupled to the components of system 10. Some of the internal components of controller 300 are described in further detail below with regarding to FIG. 3.

Robot arm 100 may include proximal end 102 coupled to a base of robot arm 100, distal end 104 configured to be coupled to, or integrated with, power drill unit 200, and a plurality of links and joints extending between proximal end 102 and distal end 104. Moreover, robot arm 100 may be equipped with built-in joint torque sensors allowing robot arm to operate in a collaborative mode. As shown in FIG. 1, power drill unit 200 may be mounted on distal end 102 of robot arm 100 at an angle of, e.g., 30° relative to the longitudinal axis of the last robot axis of robot arm 100. Robot arm 100 may be configured to automatically control and position power drill unit 150 at a desired position relative to the desired entry point of the anatomic structure, such that drill bit 200 may be positioned at the desired entry point of the anatomic structure. For example, robot arm 100 may be programmed to determine the entry point and/or trajectory of drill bit 200 for a predetermined surgical procedure.

Additionally or alternatively, robot arm 100 may be teleoperated via a master console operatively coupled to robot arm 100, such that robot arm 100 replicates movement at the master console. Robot arm 100 may be constructed, for example, as described in U.S. Pat. No. 11,344,372 and/or U.S. Patent Appl. No. 2022/0361896 to Bette, the entire contents of each of which are incorporated herein by reference. In some embodiments, robot arm 100 may be a KUKA LBR Med 7 R800 (made available by KUKA Robotics, Augsburg, Germany) adapted to medical requirements. For example, robot arm 100 may have a payload of 5 to 10 kg, preferably 7 kg, featuring position accuracy of ±0.15 mm and joint redundancy (7 degrees of freedom). As will be understood by a person having ordinary skill in the art, robot arm 100 may be another robot arm constructed in a manner within the ordinary skill in the art.

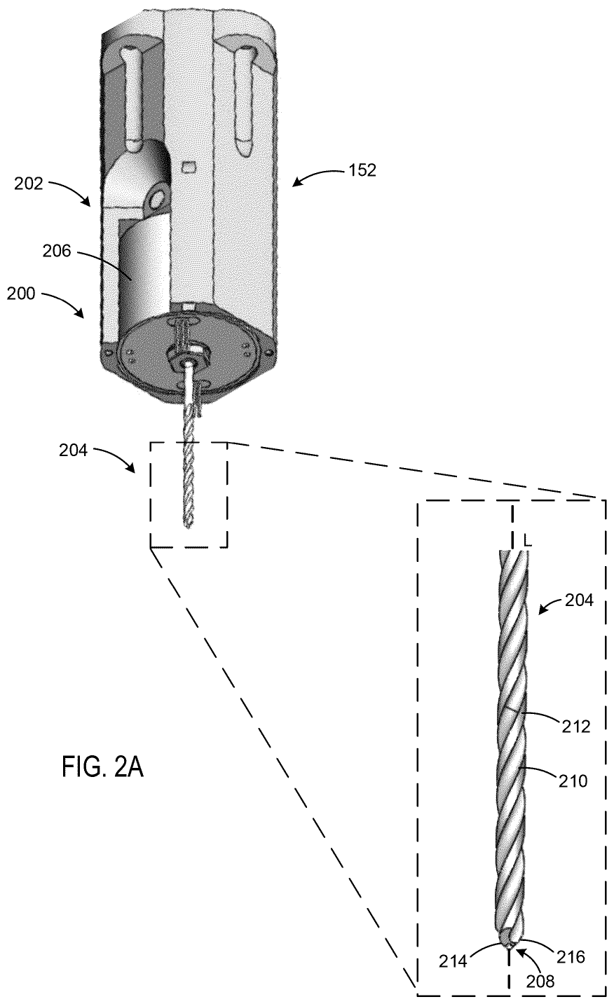

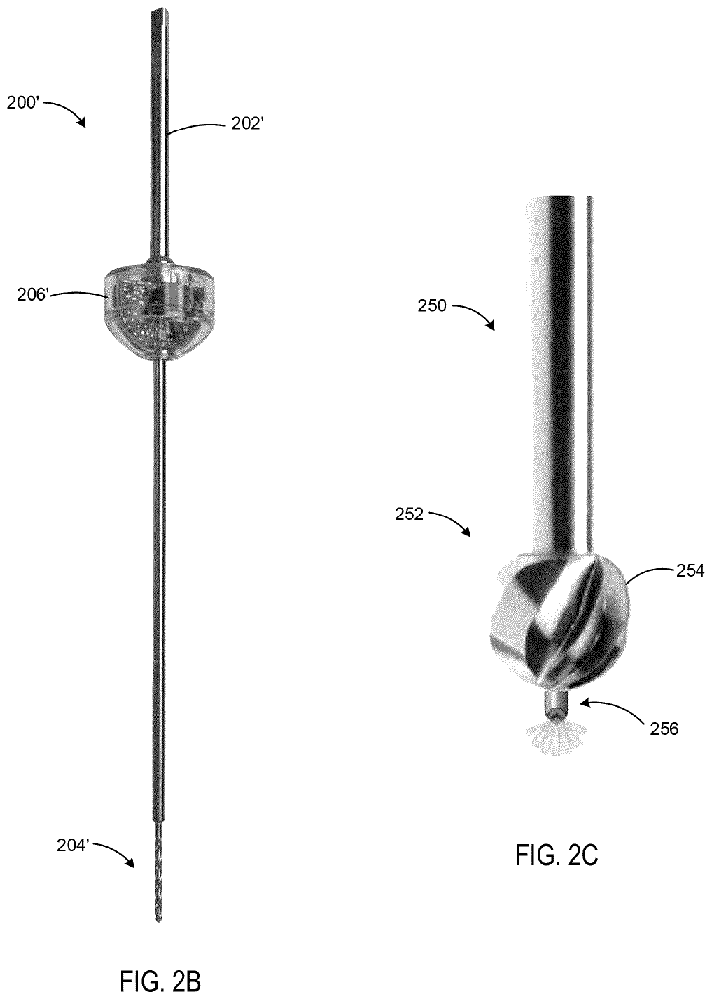

Power drill unit 150 may be coupled to the distal end of robot arm 100, and may have a sleeve, e.g., chuck 152, configured to receive a proximal end drill bit 200, such that power drill unit 150 transmits rotary motion to drill bit 200, as described in detail below. For example, power drill unit 150 may be configured to deliver perform drilling and screwing tasks, e.g., by delivering 1.5 Nm nominal torque and a maximum speed of 922 rpm (rounds per minute). Chuck 152 of power drill unit 150 may include components that establish electrical connection to drill bit 200, e.g., first and second electrodes 210 and 212 of drill bit 200 described in further detail below with regard to FIG. 2A, thus enabling controller 300 to generate an appropriate warning signal. Those components may include an electric generator operatively coupled to controller 300. The electric generator is electrically connected to drill bit 200, and is suitable for applying one or more voltages across the first and second electrodes of drill bit 200, e.g., via the conductor of drill bit 200. Controller 300 may be programmed to determine a measurement parameter related to the electrical characteristic based on a measurement electric current(s) induced by the applied voltage(s), and for emitting the warning signal corresponding to the measurement parameter. The measurement parameter may in particular be a voltage, an intensity of the electric current, conductivity or resistivity, or may be the result of processing one or more measurement electric currents, such as by integration, averaging, or the like, or may be the result of frequency analysis.

Referring now to FIG. 2A, drill bit 200 is described. As shown in FIG. 2A, drill bit 200 may include proximal end 202 sized and shaped to be received by power drill unit 150, e.g., chuck 152 of power drill unit 150, housing portion 206 disposed at a proximal region of drill bit 200 and sized and shaped to house electrical components for measuring electrical conductivity, distal end 204 configured to contact and penetrate, e.g., tissue and/or bone, and a conductor for electrically coupling drill bit 200 to power drill unit 150. Distal end 204 of drill bit 200 is configured to penetrate an anatomic portion, such as a region that includes a vertebra and surrounding tissue. As is well understood in the surgical arts, it is important to ensure precise positioning of drill bit 200 to avoid damaging or transiting the inner layer of cortical bone that delimits the foramen, or the outer layer, of cortical bone near the nerve endings. System 10 preferably is configured to emit a warning signal that varies as a function of the sensed electrical characteristic by drill bit 200 when it is moved within an anatomic portion, and to modify, e.g., arrest or slow down, penetration by drill bit 200 if drill bit 200 approaches an anatomic portion that should not be penetrated, e.g., a breach condition such as a spinal canal perforation. Drill bit 200 operates analogously to the hand tool described in U.S. Pat. No. 7,580,743, the entire contents of which are incorporated herein by reference, which device is commercially available from the assignee of the present application under the tradename PediGuard®. Accordingly, drill bit 200 may provide data including, for example, tension (in Volts), tissue electrical resistivity (in Ohms), tissue electrical conductivity (in Siemens), simple moving average (SMA), weighted moving average (WMA), exponential moving average (EMA), polynomial regression data, and filtered signals with attenuation/amplification such as a transfer function (hysteresis) and/or an analog to binary/trinary transformation. Drill bit 200 also may constitute an implant to be placed in the anatomical structure, such as a screw, and in particular a pedicle screw.

Referring still to FIG. 2A, drill bit 200 extends along longitudinal axis L between proximal end 202 and distal end 204, forming tip 208 for penetrating bony structure. Drill bit 200 generally has a cylindrical external surface of circular cross-section extending along longitudinal axis L and includes one or more spiral cutting edges that extend proximally from tip 208. The body of drill bit 200 could, however, have any other shape, in particular cylindrical with a polygonal or other cross-section. Preferably, drill bit 200 may be a threaded drill bit having a pyramidal tip embedding a conductivity sensor and a threaded shaft configured to continuously maintain the contact between drill bit 200 and the anatomic structure and avoid unwanted motion along the drilling axis. Drill bit 200 comprises first electrode 210, cylindrical and of conductive material, extending inside drill bit 200 parallel to longitudinal axis L. In particular, first electrode 210 is arranged in a central bore of drill bit 200 and extends coaxially to longitudinal axis L up to a free end having first contact surface 214, which is flush with the external surface of drill bit 200 at tip 208.

Drill bit 200 also includes second electrode 212, annular and of conductive material, extending along longitudinal axis L around first electrode 210. In particular, second electrode 212 is formed by a portion of drill bit 200 itself, made in this case of a conductive material. Second electrode 212 has second contact surface 216 composed of a cylindrical portion parallel to longitudinal axis L and corresponding to a lateral surface of drill bit 200, and an annular portion transverse to longitudinal axis L corresponding to a distal surface of drill bit 200.

A layer of electrically insulating material is interposed between first electrode 210 and second electrode 212 such that first contact surface 214 and second contact surface 216 can come into contact, at a distance from one another, with the anatomic portion during penetration of drill bit 200 into the anatomic portion. It should be understood, however, that the invention is not limited to the embodiment illustrated by drill bit 200, and other shapes are possible, such as, for example, that first electrode 210 and second electrode 212 are not arranged coaxially but may be formed from a rod of conductive material inserted into drill bit 200. Furthermore, first electrode 210 and second electrode 212 each may have a point-like or other contact surface 214, 216, flush with the lateral surface or distal surface of drill bit 200. Alternatively, drill bit 200 could support two or more first electrodes 210 and two or more second electrodes 212.

FIG. 2B illustrates an alternative drill bit for use with robot arm 100. Drill bit 200′ may be constructed similar to drill bit 200 with similar components having like-prime reference numerals. For example, drill bit 200′ includes proximal end 202′ sized and shaped to be received by a power drill unit for transmission of rotary motion to drill bit 200′, distal end 204′ configured to contact and penetrate an anatomic structure, e.g., tissue and/or bone, and having the sensing capabilities described herein, and housing portion 206′ sized and shaped to electrical components for measuring electrical conductivity.

Referring now to FIG. 2C, the sensing capabilities described herein may be applied to a surgical burr, e.g., for drilling during a bone resection (laminectomy) procedure. As shown in FIG. 2C, distal end 252 of surgical burr 250 may include drilling portion 254 configured to contact and penetrate an anatomic structure, e.g., tissue and/or bone, and sensing pin 256 having the sensing capabilities described herein. The proximal end of surgical burr 250 (not shown) may be sized and shaped to be received by a power drill unit for transmission of rotary motion to surgical burr 250. As will be understood by a person having ordinary skill in the art, drilling portion 254 may have various shapes and/or types selected based on the surgical procedure. For example, drilling portion 254 may have a ball shape, an acorn shape, a match head shape, a tapered shape, a reverse tapered shape, an oval shape, a cylinder shape, a twist shape, etc., and/or may be a fluted type, a diamond type, a coarse diamond type, an extra-coarse diamond type, a carbide type, a symmetric type, etc.

Referring now to FIG. 3, components that may be included in controller 300 used in conjunction with robot arm 100, power drill unit 150, and drill bit 200 are described. Controller 300 may be operatively coupled to the electric components of robot arm 100, power drill unit 150, and drill bit 200, such that controller 300 may receive signals indicative of electrical conductivity measurements from drill bit 200, e.g., based on electrical impedance of the surrounding tissue and/or bone, detect a condition, e.g., a breach during a surgical drilling procedure such as a perforation of the spinal canal, based on the signals, and instruct power drill unit 150 to cease rotation of drill bit 200.

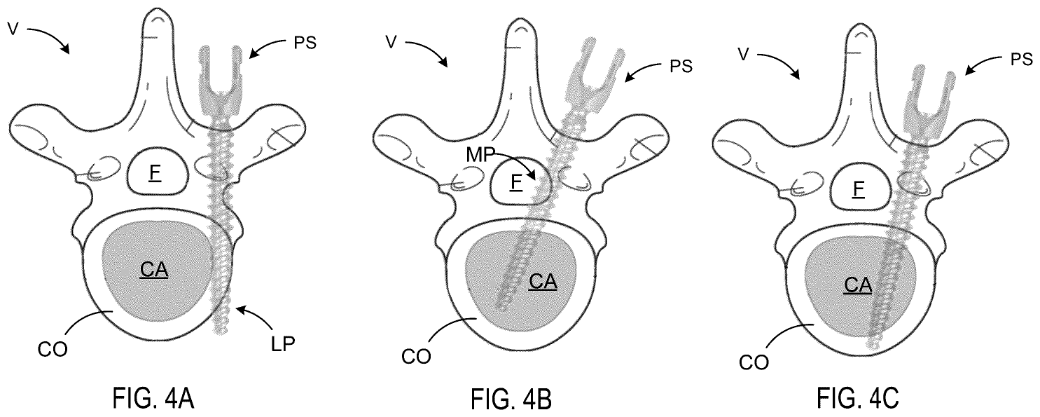

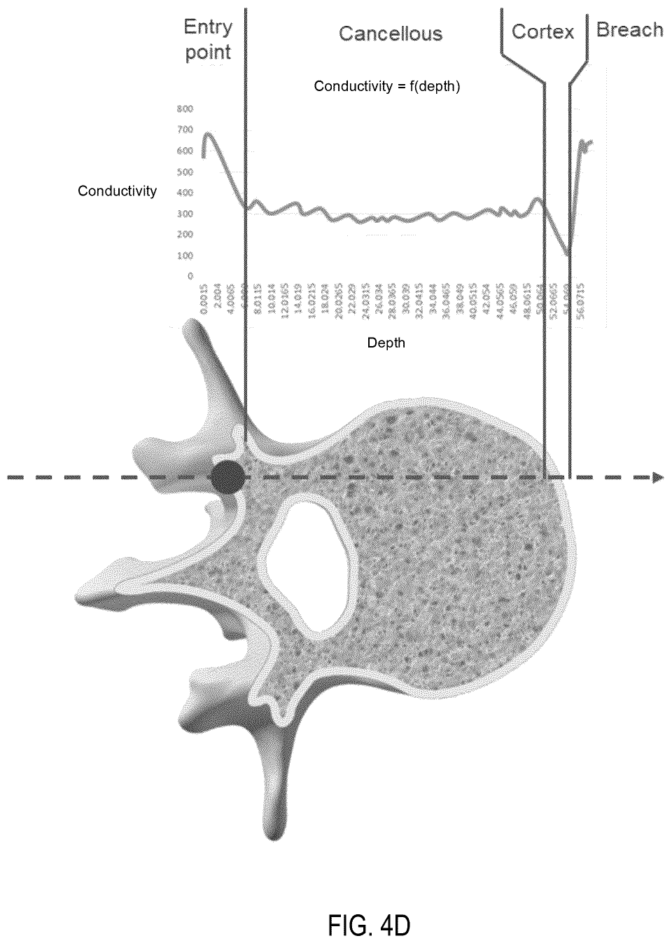

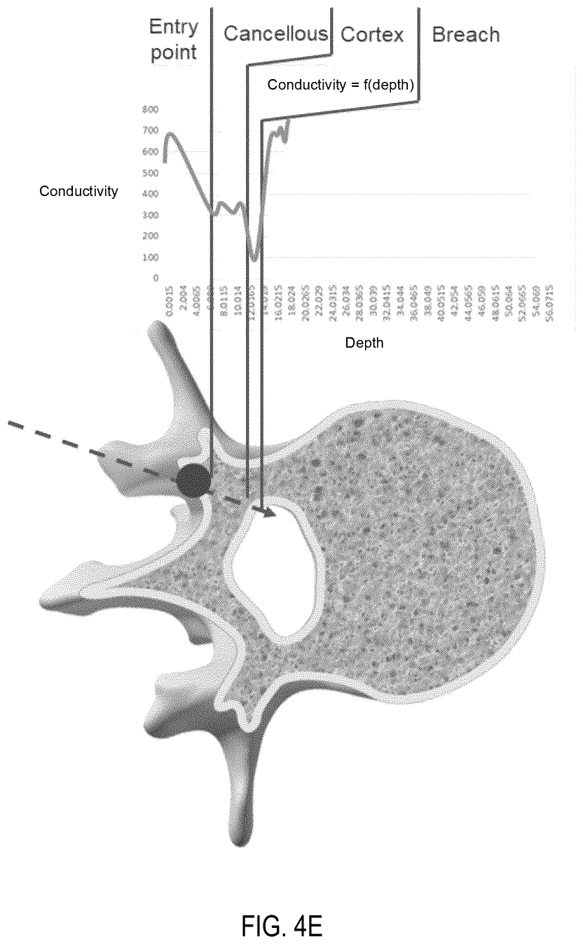

For example, FIG. 4A illustrates insertion of pedicle screw PS into vertebra V leading to lateral/anterior perforation LP, e.g., a transition to the outer layer of cortical bone CO near the nerve ending. As shown in FIG. 4D, as a drill bit is inserted along the trajectory denoted by the dashed line, electrical conductivity detected by the drill bit decreases as the drill bit passes through the entry point and into cancellous bone, decreases even further as the drill bit enters the cortical bone (cortex), and significantly increases as the drill bit exits the outer layer of the cortical bone, indicating a lateral/anterior perforation. FIG. 4B illustrates insertion of pedicle screw PS into vertebra V leading to medial/canal perforation MP, e.g., a transition to the inner layer of cortical bone CO and into cancellous bone CA delimiting foramen F. As shown in FIG. 4E, as a drill bit is inserted along the trajectory denoted by the dashed line, electrical conductivity detected by the drill bit decreases as the drill bit passes through the entry point and into cancellous bone, decreases even further as the drill bit enters the cortical bone (cortex) surrounding the foramen, and significantly increases as the drill bit exits the cortical bone and passed into the foramen, indicating a medial/canal perforation. FIG. 4C illustrates a well-placed pedicle screw PS into vertebra V.

Referring again to FIG. 3, controller 300 may include one or more processors 302, communication circuitry 304, power supply 306, user interface 308, and/or memory 310. Memory 310 may be RAM, ROM, Flash, or other known memory, or some combination thereof, and preferably includes storage in which data may be selectively saved. For example, programmable instructions may be stored to execute algorithms for detecting a breach, e.g., perforation, or near breach of the elongated drilling portion during a surgical drilling procedure into bone. One or more electrical components and/or circuits may perform some of or all the roles of the various components described herein. Although described separately, it is to be appreciated that electrical components need not be separate structural elements. For example, controller 300 and communication circuitry 304 may be embodied in a single chip. In addition, while controller 300 is described as having memory, a memory chip(s) may be separately provided.

Controller 300 may incorporate processor 302, which may consist of one or more processors and may be a general purpose processor, a digital signal processor (DSP), an application specific integrated circuit (ASIC), a field programmable gate array (FPGA) or other programmable logic device, discrete gate or transistor logic, discrete hardware components, or any suitable combination thereof designed to perform the functions described herein. The controller also may be implemented as a combination of computing devices, e.g., a combination of a DSP and a microprocessor, a plurality of microprocessors, one or more microprocessors in conjunction with a DSP core, or any other such configuration.

Controller 300, in conjunction with firmware/software stored in the memory may execute an operating system (e.g., operating system 326), such as, for example, Windows, Mac OS, Unix or Solaris 5.10. Controller 300 also executes software applications stored in the memory. In one non-limiting embodiment, the software comprises, for example, Unix Korn shell scripts. In other embodiments, the software may be programs in any suitable programming language known to those skilled in the art, including, for example, C++, PHP, or Java.

Communication circuitry 304 may include circuitry that allows controller 300 to communicate with the electronic components of robot arm 100 and/or power dill unit 150, e.g., the power supply, the electric generator, an alarm system, and the brake mechanism of power drill unit 150, with the electronic components of drill bit 200, e.g., the electrodes, the electric generator, and/or the electric processing device, and optionally with the electronic components of external computing device 160, e.g., the display. Communication circuitry 304 may be configured for wired and/or wireless communication over a network such as the Internet, a telephone network, a Bluetooth network, and/or a WiFi network using techniques known in the art. Communication circuitry 304 may be a communication chip known in the art such as a Bluetooth chip and/or a WiFi chip. Communication circuitry 304 permits controller 300 to transfer information, such as signals indicative of a breach or near breach associated with spinal drilling, locally and/or to a remote location such as a server. Power supply 306 may be designed to supply power to the components of robot arm 100, power drill unit 150, and/or drill bit 200.

User interface 308 may be used to receive inputs from, and/or provide outputs to, a user. For example, user interface 308 may provide information to the user on the detection of a breach, e.g., a spinal canal perforation, or near breach during the drilling procedure. User interface 308 further may include an audible device and/or volume control to selectively increase or decrease an audio output. User interface 308 may include 308 a touchscreen, switches, dials, lights, an LED, an LED matrix, other LED indicators, or other input/output devices for receiving inputs from, and/or providing outputs to, a user. In other embodiments, user interface 308 may integrated with remote, external computing device 160 communicatively connected to the components of system 10 via the communication circuitry 304. User interface 308 also may be a combination of elements on the power drill unit, the robot arm, and/or the external computing device.

Memory 310, which is one example of a non-transitory computer-readable medium, may be used to store operating system (OS) 326, robot arm interface module 312, power drill interface module 314, electric generator interface module 316, conductivity sensing module 318, condition detection module 320, alert generation module 322, and display interface module 324. The modules are provided in the form of computer-executable instructions that may be executed by processor 302 for performing various operations in accordance with the disclosure. Instructions may be stored, for example, for executing statistical perforation detection algorithms associated with the breach detection based on changes in electrical conductivity as described herein, as well as other breach detection algorithms as described in U.S. Pat. No. 11,344,372 to Bourlion or U.S. Patent Appl. Publ. No. 2022/0361896 to Bette, the entire contents of each of which are incorporated herein by reference.

Robot arm interface module 312 may be executed by processor 302 for determining the entry point and/or trajectory of drill bit 200 for a predetermined surgical procedure. Moreover, robot arm interface module 312 may generate and transmit one or more signals to robot arm 100 to cause robot arm 100 to position power drill unit 150 at a desired position relative to the desired entry point of the anatomic structure, such that drill bit 200 may be positioned at the desired entry point of the anatomic structure.

Power drill interface module 314 may be executed by processor 302 for sending one or more command signals to power drill unit 150 to actuate drill bit 200, e.g., by causing rotation of the elongated drilling portion of drill bit 200, to thereby penetrate the anatomic portion in accordance with one or more preset drilling parameters, e.g., feed rate, rotation speed, etc. In addition, power drill interface module 314 may send one or more signals to power drill unit 150 to cause power drill unit 150 to cease transmission of rotary motion and arrest advancement of the elongated drilling portion of drill bit 200 relative to the anatomic portion being penetrated, e.g., based on the warning signal generated by alert generation module 322, as described in further detail below. For example, power drill interface module 314 may cause power drill unit 150 to stop rotation of the elongated drilling portion of drill bit 200, to thereby cease penetration of the anatomic portion. Alternatively, power drill interface module 314 may cause power drill unit 150 to modify rotation of the elongated drilling portion of drill bit 200, e.g., the speed of rotation, based on the warning signal generated by alert generation module 322, to thereby slow down penetration of the anatomic portion.

Electric generator interface module 316 may be executed by processor 302 for causing the electric generator of drill bit 200 to apply one or more voltages across the first and second contact surfaces, e.g., electrodes, of drill bit 200 during penetration of the anatomic portion by the elongated drilling portion.

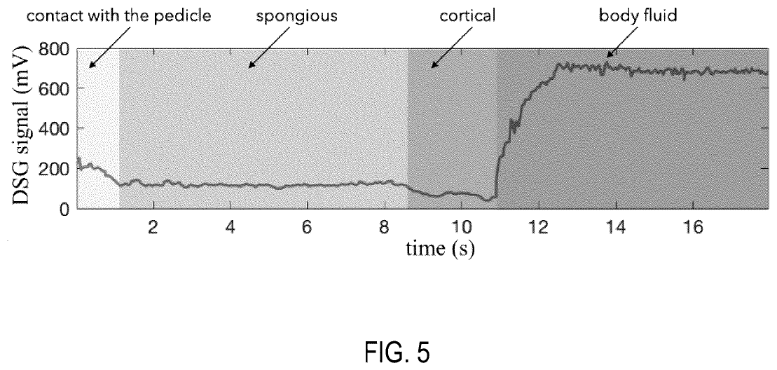

Conductivity sensing module 318 may be executed by processor 302 for receiving one or more signals from the electrodes of drill bit 200 indicative of measured electrical conductivity as the elongated drilling portion penetrates the anatomic portion. Specifically, conductivity sensing module 318 may determine a measurement parameter related to the electrical characteristic, e.g., voltage, an intensity of the electric current, conductivity or resistivity, based on a measurement electric current(s) induced by the applied voltage(s). Accordingly, conductivity sensing module 318 may measure the electrical conductivity, e.g., based on electrical impedance of the tissue and/or bone surrounding the distal tip of the elongated drilling portion as it penetrates the anatomic portion in real-time, which may be used to distinguish the different layers drill bit 200 passes through during the drilling process. For example, as shown in FIG. 5, which illustrates exemplary conductivity signals (DSG signal) acquired during a vertebra drilling process over time, the conductivity signal is low in the cortical layers compared to the conductivity signal detected in the cancellous layers, and increases significantly once drill bit 200 intersects body fluid. Accordingly, these tissue discrimination characteristics are suitable for the detection of perforations (lateral or medial).

Condition detection module 320 may be executed by processor 302 for detecting a condition, e.g., a breach during a surgical drilling procedure such as a spinal canal perforation, based on signals indicative of the measured electrical conductivity by conductivity sensing module 318. For example, based on the signals indicative of electrical conductivity measurements, condition detection module 320 may detect a breach condition such as transition to the inner layer of cortical bone delimiting the foramen, or transition to the outer layer of cortical bone near the nerve endings. A goal may be to arrest advancement of the elongated drilling portion into the anatomic portion being penetrated when rapid variations in the signals are observed, and a delay of more than one second may cause a breach at the end of drilling. Accordingly, condition detection module 320 may execute one or more algorithms, e.g., statistical perforation detection algorithms, stored therein to mathematically detect when changes in electrical conductivity, e.g., as the elongated drilling portion penetrates into the anatomic portion, satisfy one or more predetermined conditions, as described in further detail below.

Alert generation module 322 may be executed by processor 302 for generating a warning signal when condition detection module 320 detects a condition, and optionally causing an alarm system operatively coupled to, e.g., robot arm 100, power drill unit 150, drill bit 200, or external computing device 160 carrying controller 300, to emit a warning, e.g., an audible, visual, and/or tactile warning, based on the warning signal. For example, alert generation module 322 may cause the alarm system, e.g., a speaker, to cmit an audible warning signal frequency-modulated and possibly intensity-modulated, which may vary based on the change in electrical conductivity detected by condition detection module 320. Moreover, alert generation module 322 may generate one or more signals indicative of the type of tissue/bone that the distal end of drill bit 200 is currently in in real-time as drill bit 200 penetrates the anatomic structure, such that controller 300 may control the operational drilling parameters based on the tissue/bone that the distal end of drill bit 200 is in, e.g., via robot arm interface module 312 and/or power drill interface module 314. For example, if the signal generated by alert generation module 322 indicates that drill bit 200 is in cancellous bone, controller 300 may cause power drill unit 150 to operate in a normal mode under normal drilling parameters; if the signal generated by alert generation module 322 indicates that drill bit 200 is in cortical bone, controller 300 may cause power drill unit 150 to operate in a caution mode, e.g., with reduced feed rate and rotation speed; and if the signal generated by alert generation module 322 indicates that a breach condition is detected, controller 300 may cause power drill unit 150 to cease transmission of rotary motion and arrest advancement of the elongated drilling portion of drill bit 200 relative to the anatomic portion being penetrated. For example, power drill interface module 314 may send one or more signals to power drill unit 150 to cause power drill unit 150 to cease transmission of rotary motion to drill bit 200, based on the warning signal generated by alert generation module 322. Accordingly, the warning signal may cause controller 300 to temporarily disable system 10. In some embodiments, the user may override controller 300 and continue drilling despite the presence of the warning signal to auto-stop drill bit 200, e.g., by actuating robot arm 100 and/or power drill unit 150 teleoperatively via one or more actuators on a master console operatively coupled to robot arm 100 and/or power drill unit 150.

Display interface module 324 may be executed by processor 302 for rendering and transmitting data to a display operatively coupled to controller 300, e.g., disposed on user interface 308 and/or remote computing device 160, for displaying information associated with the transmitted data. For example, display interface module 324 may cause information indicative of the conductivity as determined by conductivity sensing module 318 to be displayed.

As described above, controller 300 may be programmed to execute one or more statistical perforation detection algorithms detect when changes in electrical conductivity satisfy one or more predetermined conditions. Specifically, the statistical perforation detection algorithm described herein is a probabilistic perforation detection algorithm referred herein as a Bayesian Online Perforation Detector (BOPD) algorithm, based on a Bayesian Online Change Point Detection (BOCPD) approach, which implement probabilistic and recursive methods for accurate detection of an abrupt change in a time series, e.g., at “changepoints” (CPs). Changepoints are time instants when the probability distribution of a time series changes. BOCPD algorithms have been found to be one of the best performing methods for offline and online CP detection in the finance and environmental industries.

In the BOCPD approach, x=(x₁, x₂ . . . , xₙ)^{T} denotes the data samples observed over time. In the BOPD algorithm, x is univariate, but the algorithm may be extended to multivariate cases. Moreover, in the BOCPD approach, x_{1:t} denotes the data samples observed between the initial time instant 1 and time instant t. CPs occur at unknown time instants between the initial time instant t=1 and final time instant t=n. These CPs divide x into production partitions ρ. For each partition ρ, it is assumed that the data samples within that partition are independent and identically distributed and sampled from some probability distribution P(xₜ|ηₚ), where the parameters η_{ρ} are also considered independent and identically distributed. BOCPD relies on computing the probability distribution of an intermediate variable, the “run length”, i.e., the time since the most recent CP occurred. The run length at time instant t is denoted as rₜ. Observing rₜ=0 indicates that a CP occurred at time instant t. Arbitrarily, the first data sample observed is considered as an additional CP occurring at initial time instant t=1, implying that rₜ=0. xₜ^{r} denotes the subset of observations associated with the run rₜ. Between two consecutive time instants, the run length can either reset to 0 if a CP occurred or be incremented by 1 if no CP occurred (Equation 1):

$${r}_{t} = \{ \begin{matrix}0 & if a CP occured \\ {r}_{t - 1} + 1 & otherwise\end{matrix}$$

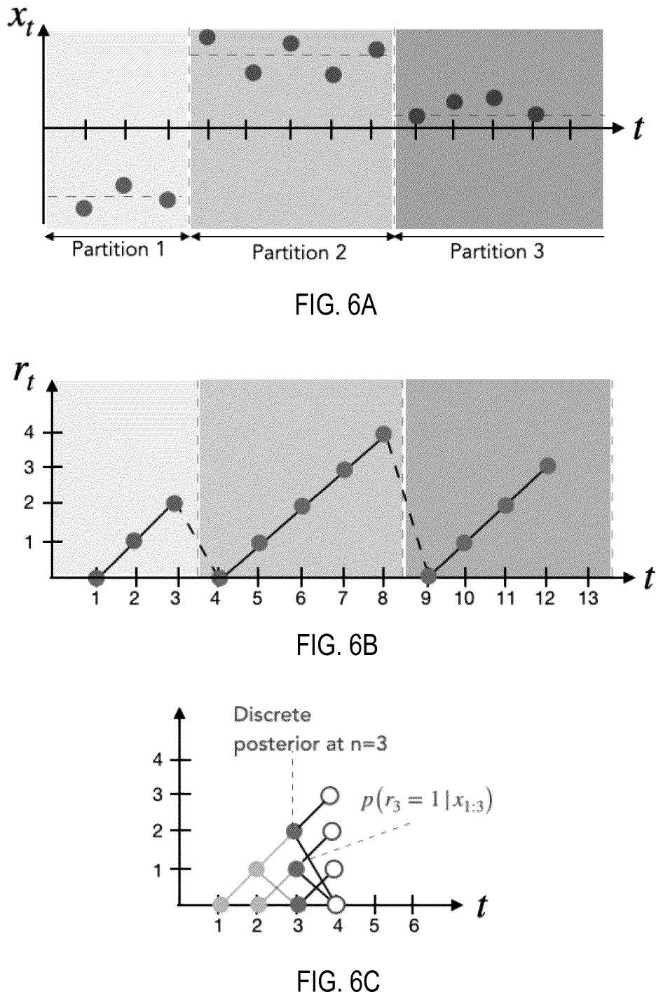

A hypothetical univariate signal x is plotted in FIG. 6A, which illustrates three sets of data partitions, each with similar distributions/trends. As shown in FIG. 6A, significant changes in the distribution of x occur at time instant 4 and time instant 9. At these time instants, rₜ resets to 0, as shown in FIG. 6B. Specifically, FIG. 6B illustrates evolution of the most probable run lengths over time.

Once a new observation xₜ is observed, we determine whether run length rₜ is incremented or reset to 0 by computing the posterior probability distribution P(rₜ|x_{1:t}). In other words, BOCPD aims at finding at every time instant the run length rₜ that best matches the data x_{1:t} observed so far. In Bayesian terms, for every time instant t, one wants to find

$${r}_{t} = {arg max}_{r} P ( {r}_{t} = r \vert {x}_{1 : t} )$$

where (Equation 2):

$$P ( {r}_{t} \vert {x}_{1 : t} ) = \frac{P ( {r}_{t} , {x}_{1 : t} )}{P ( {x}_{1 : t} )}$$

Since in Equation 2 only the joint distribution over the run length and observed data P(rₜ,x_{1:t}) depends on rₜ, one can focus on this distribution and rewrite it recursively (Equation 3):

$$P ( {r}_{t} , {x}_{1 : t} ) = {\sum }_{{r}_{t - 1}} P ( {r}_{t} \vert {r}_{t - 1} ) P ( {x}_{t} \vert {r}_{t - 1} , {x}_{t}^{r} ) P ( {r}_{t - 1} , {x}_{1 : t - 1} )$$

As shown in Equation 3, P (rₜ,x_{1:t}) can be expressed as a function of P(rₜ₋₁,x_{1:t-1}). This provides a recursive message-passing algorithm for the joint distribution over the current run length and the data, assuming two other factors are computed: the CP prior and the posterior predictive. FIG. 6C, which is a Bayesian visualization of the run length-associated probabilities, illustrates this message-passing algorithm. As shown in FIG. 6C, the circles represent run-length hypotheses, and the lines between the circles show the recursive transfer of probability mass between consecutive time instants. A probability distribution is associated with each circle, i.e., run-length hypothesis. For each hypothesis, the probability distribution parameters are obtained by updating the initial parameters prior to every data sample observed since the beginning of the run. In FIG. 6C, solid lines indicate that the run length grows at the next time instant, and the dotted lines indicate that the current run is truncated and the run length drops to 0.

P(rₜ|rₜ₋₁) is the prior over rₜ given rₜ₋₁. This prior is non-zero at only two outcomes, since rₜ only changes to 2 possible values, as shown in Equation 1. This provides computational efficiency to the method and the prior simplifies to Equation 4:

$$P ( {r}_{t} \vert {r}_{t - 1} ) = \{ \begin{matrix}ℋ ( {r}_{t - 1} + 1 ) & if {r}_{t} = 0 \\ 1 - ℋ ( {r}_{t - 1} + 1 ) & if {r}_{t} = {r}_{t - 1} + 1 \\ 0 & otherwise\end{matrix}$$

where [symbol] is referred to as a hazard function. A constant hazard function means that the probability of a changepoint occurring does not depend on the run length and could happen equally likely at any time.

P(xₜ|rₜ₋₁,xₜ^{r}) is the posterior predictive, i.e., the probability that the most recent datum belongs to a given run. While computing this posterior predictive may be challenging, using conjugate exponential models allows for more computational efficiency since they provide a closed-form expression of the posterior predictive. For example, exponential families, which include many of the most common distributions, are good candidates and allow sequentially updating the distribution parameters as new data are observed.

BOCPD does not explicitly specify a determination criterion to declare a CP at a specific time instant. Identifying a CP after a single sample of a new distribution may be challenging in certain cases, making it necessary to “wait” for N samples and evaluate the probability of a change happening N samples prior. Therefore, in terms of run length, detecting that rₜ drops to 0 after a new observation is not an efficient criterion for accurate CP detection. Instead, we detect that rₜ drops in a [0, N] range.

Accordingly, by building on BOCPD. the proposed BOPD approach is illustrated by the following perforation detection algorithm using the electrical conductivity signal:

Input: xₜ, DSG (electrical conductivity) signal in mV
Output: Alert, flag used to stop drilling

| | |
|---|---|
| 1: | Initialization: P(r₁ = 0) ← 1, DetectionFlag ← False, |
| 2: | for xₜ do |
| 3: | Calculate posterior predictive: |
| | P(xₜ\|rₜ₋₁, xₜ^{r}) |
| 4: | Calculate growth probability: |
| | P(rₜ = rₜ₋₁ + 1, x_{1:t}) = (1 − λₚᵣᵢₒᵣ⁻¹)P(xₜ\|rₜ₋₁, xₜ^{r})P(rₜ₋₁, x_{1:t-1}) |
| 5: | Calculate changepoint probability: |
| | P(rₜ = 0, x_{1:t}) = Σᵣ_{_{t−1}}λₚᵣᵢₒᵣ⁻¹ P(xₜ\|rₜ₋₁, xₜ^{r})P(rₜ₋₁, x_{1:t-1}) |
| 6: | Calculate marginal probability: |
| | P(x_{1:t}) = Σᵣ_{ₜ}P(rₜ, x_{1:t}) |
| 7: | Calculate run length distribution: |
| | P(rₜ\|x_{1:t}) = P(rₜ, x_{1:t})/P(x_{1:t}) |
| 8: | Find: |
| | ${r}_{t} = {arg max}_{r} P ( {r}_{t} = r \vert {x}_{1 : t} )$ |
| 9: | Check that run length has exceeded the detection threshold once: |
| 10: | if rₜ > threshold then |
| 11: | DetectionFlag ← True |
| 12: | end if |
| 13: | Evaluate whether a change occurred: |
| 14: | if DetectionFlag & rₜ < threshold then |
| 15: | Alert ← True |
| 16: | end if |
| 17: | Update distribution parameters |
| 18 | end for |

Regarding the practical implementation, the following hypotheses are made:
A CP is declared if the run length drops in a [0, 19] range;
P(rₜ|rₜ₋₁) is assumed to be constant for both simplicity and since no other prior information is available. Therefore, in Equation 4, [symbol] (rₜ)=λ⁻¹, where λ is a parameter to adjust the algorithm sensitivity. A value of λ=250 is empirically established;
As a generic assumption, normal distribution unknown mean and variance is assumed as a likelihood. The corresponding conjugate prior (i.e., if the posterior ends up being in the same distribution as the prior based on the chosen prior) is a Normal-Inverse-Gamma distribution. The resulting posterior distribution is a generalized Student's T distribution with center μₜ, precision

$$Λ = \frac{{α}_{t} {κ}_{t}}{{β}_{t} ( {κ}_{t} + 1 )}$$

and degree of freedom 2αₜ (Equation 5):

$$P ( {x}_{t} \vert {r}_{t - 1} , {x}_{t}^{r} ) = {t}_{2 {α}_{t}} ( {x}_{t} \vert {μ}_{t} , \frac{{β}_{t} ( {κ}_{t} + 1 )}{{κ}_{t} {α}_{t}} )$$

The Student's T distribution may be used to obtain the probability of the new observation. Parameters μₜ, κₜ, αₜ, and βₜ are initialized with pre-perforation distribution priors μ₀, κ₀, α₀, and β₀ at time t=0. Then, at each time instant t, we want to model a new possible run length of 0, corresponding to the possibility of a CP. In this case, posterior predictive is computed using post-perforation distribution priors μₚᵣᵢₒᵣ, κₚᵣᵢₒᵣ, αₚᵣᵢₒᵣ, and βₚᵣᵢₒᵣ. These priors may be different from the initial values μ₀, κ₀, α₀, and β₀ to encompass the knowledge that a strong rise in the conductivity signal is expected upon perforation. Exemplary values of the distribution parameters priors used in the BOPD are provided in Table 1 below.

TABLE 1

| Distribution | Pre-Perforation | Post-Perforation |
|---|---|---|
| Parameter | Distribution | Distribution |
| μ | μ₀ = 800 | μₚᵣᵢₒᵣ = 10000 |
| κ | κ₀ = 1 | κₚᵣᵢₒᵣ = 10 |
| α | α₀ = 10⁻⁶ | αₚᵣᵢₒᵣ = 10⁻⁶ |
| β | β₀ = 0.6 | βₚᵣᵢₒᵣ = 0.6 |

At each time instant t, the growth for already existing runs is modeled. This is achieved by computing the posterior predictive using parameters μₜ, κₜ, αₜ, and βₜ for each run length value before updating these values with the latest datum observed:

$${μ}_{t + 1} = \frac{{κ}_{t} {μ}_{t} + {x}_{t + 1}}{{κ}_{t + 1}} \qquad Equation 6$$

$${κ}_{t + 1} = {κ}_{t} + 1 \qquad Equation 7$$

$${α}_{t + 1} = {α}_{t} + 0.5 \qquad Equation 8$$

$${β}_{t + 1} = {β}_{t} + \frac{{{κ}_{t} ( {x}_{t} - {μ}_{t} )}^{2}}{2 ( {κ}_{t} + 1 )} \qquad Equation 9$$

The BOPD approach allows for accurate perforation detection in real-time without requiring filtering of the electrical conductivity signal or prior calibration. Moreover, the detection threshold may be, for example, defined via user input, predetermined based on past experimental data and/or a clinical database, and/or calculated in real-time in vivo.

Although only the conductivity signal is used in the perforation detection algorithm, controller 300 may monitor and record different signals generated from various embedded sensors including, for example, torque, position (depth penetration of the drill bit), drilling angle, velocity, time, video, etc. For example, in some embodiments, drill bit 200 may include a depth sensor configured to generate one or more signals indicative of the depth of penetration into the anatomic structure by the elongated drilling portion of drill bit 200, such that controller 300 may determine the depth of penetration of the distal end of the elongated drilling portion into the anatomic portion based on the one or more signals, as described in U.S. Patent Appl. Publ. No. 2023/0095197 to Chandanson. For example, the one or more signals may be indicative of depth as measured by at least one of a linear potentiometer, a laser distance sensor, an infrared distance sensor, an ultrasonic distance sensor, a Light Detection and Ranging (LiDAR) sensor, a 3D Time-of-Flight camera, linear magnetic or hall effect encoders, a reversible linear actuator such as a lead screw, or an inductive linear position sensor. Accordingly, based on the signals indicative of electrical conductivity and penetration depth measurements, condition detection module 320 may determine that one or more additional predetermined conditions are satisfied, e.g., when a predetermined maximum depth of penetration is reached, when a predetermined depth of penetration a predetermined distance beyond the depth when a breach condition is detected is reached, e.g., 2 mm beyond the breach condition depth such that the cortex is completely perforated so that a bone screw may be inserted such that its first threads bite the cortical wall for a “bicortical fixation” technique, when a cancellous and/or cortical is reached, etc.

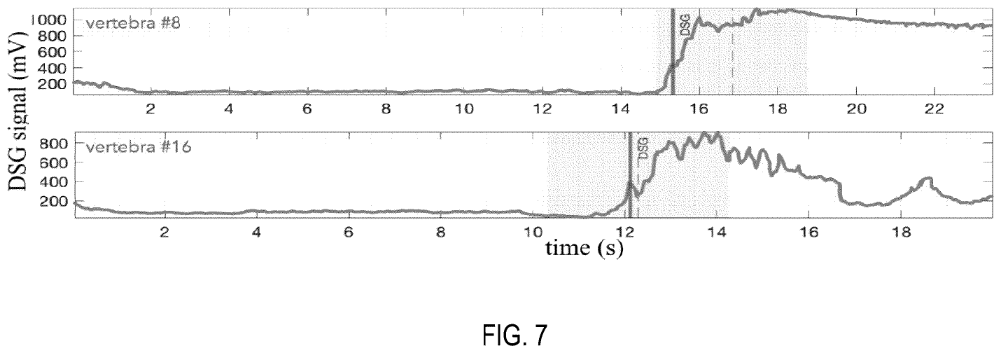

The perforation detection algorithms based on the BOPD approach described herein were validated via both numerical and experimental validations by applying the algorithm on data collected from the drilling of 80 lumbar pig vertebrae. First, the electrical conductivity signals recorded for each drilling were evaluated offline. For each signal, a surgeon defined the exact time instant of perforation by a posteriori visualizing videos recorded during the drilling, which allows grading perforation detection according to a derivation of the Gertzbein-Robbins classification. For example, under the derived Gertzbein-Robbins classification, detection of an imminent perforation before the actual point of perforation corresponds to grade “A”, while declaring a perforation less than 2 mm after the actual perforation point corresponds to grade “B”. Thus, the detection is considered acceptable if a perforation alert is raised before the right-most limit of the shaded area in the plots illustrated in FIG. 7, e.g., within 2 mm of the actual perforation point denoted by the vertical dashed lines.

For the 80 drilled vertebrae, the success rate of the perforation detection algorithm was 100%, meaning all of the perforations were detected with an error of less than 2 mm corresponding to grades “A” and “B” drillings according to the derived Gertzbein-Robbins classification of pedicle screw placement. Specifically, 25% of the drillings are classified as “A” (imminent perforation is detected before actual perforation point), and 75% as “B” (i.e., an error between 0 mm and 2 mm). In terms of accuracy, the perforation detection algorithm allows stopping of the drilling with an average error (i.e., distance from the perforation point) of 0.50 mm with a standard deviation (STD) of 0.71 mm.

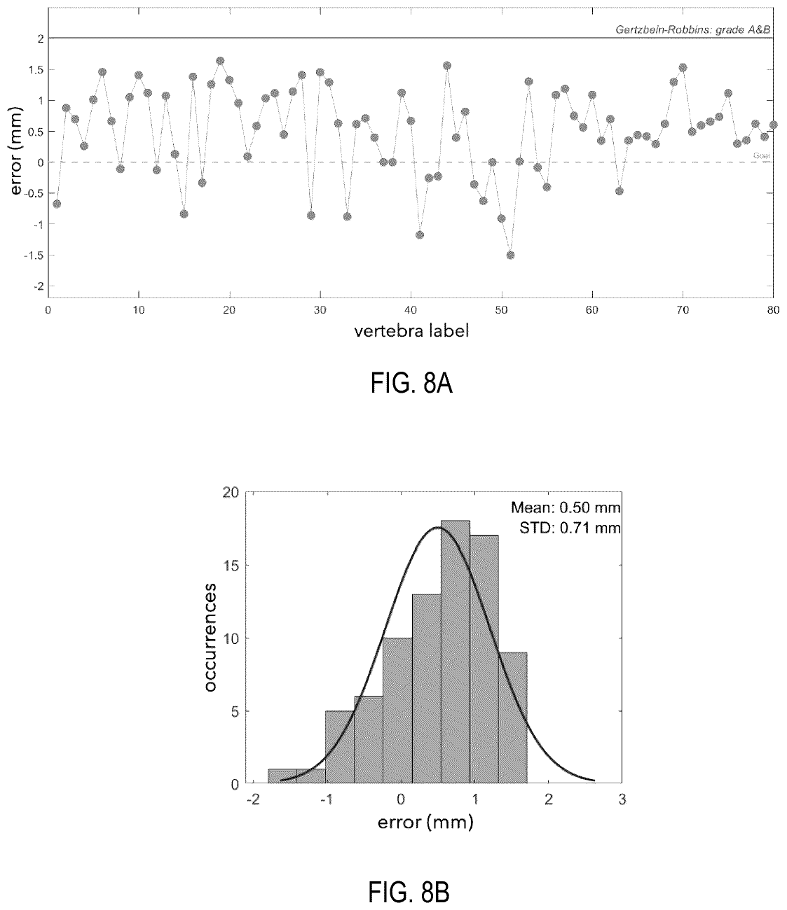

FIG. 7 illustrates two examples of electrical conductivity signals for which the perforation was detected (e.g., vertebra #8 and vertebra #16). As shown in FIG. 7, the detection of the perforation (solid vertical lines denoted “DSG”) corresponding to the abrupt change in the signal occurred even before the perforation point. The perforation detection over the whole set of the 80 performed drillings is summarized in FIGS. 8A and 8B, where FIG. 8A depicts the distance of the automatic stop to the perforation point for each drilled vertebra, and FIG. 8B shows the histogram of all the computed distances for the 80 drilled vertebrae.

The robotic setup and the perforation detection algorithm were then experimentally assessed based on the numerical evaluation and the obtained promising results in terms of accuracy and safety during the drilling procedure. The experimental procedure was identical to the one used to build the database. For this purpose, another group of 24 fresh lumbar pig vertebrae was used. The surgeon moved the drill bit to the entry point of the pedicle (by manipulating the robotic arm), and the drilling was automatically performed. The robot drilled the vertebrae in conditions close to those of an operating room, and the drilling was stopped online automatically by the perforation detection algorithm without any calibration or pre-setting procedure compared to the numerical validation procedure. Once the vertebrae were drilled, the surgeon cut the pedicles along the drilled hole to verify the location where the drilling was stopped, which showed that for all 24 drilled vertebrae, the drilling process was automatically stopped just before the perforation into the spinal canal, i.e., at the interface of the cortical bone, exhibiting a 100% success rate according to the derived Gertzbein-Robbins classification.

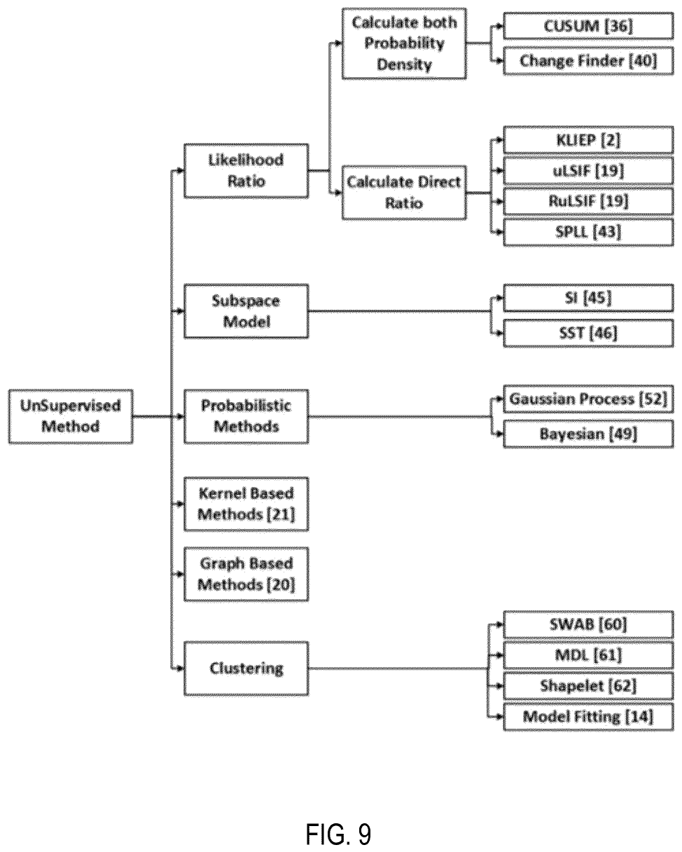

Referring now to FIG. 9, a classification of unsupervised methods for changepoint detection is provided. The following methods have been identified as potentially interesting for a vertebra drilling application. RBeast is an offline probabilistic Bayesian-based method that decomposes time series as the linear combination of basis functions, e.g., line segment or harmonics, to detect changepoints. See, e.g., K. Zhao et al., “Detecting change-point, trend, and seasonality in satellite time series data to track abrupt changes and nonlinear dynamics: A Bayesian ensemble algorithm,” Remote Sensing of Environment, vol. 232, p. 111181, October 2019, doi: 10.1016/j.rsc.2019.04.034. RBeast allows extraction of seasonal trend and location of changepoints. As new data is observed by RBeast, the probabilities associated with the following are computed: probabilities associated to possible numbers and locations of changepoints; and probabilities associated to the basis functions linear combination coefficients values. An RBeast approach may be suitable for real-time changepoint detection associated with a spinal canal breach during vertebra drilling, e.g., by choosing ad hoc basis functions and priors in the basis functions coefficients and number and location of changepoints. For example, by using a well-chosen basis function family (e.g., using prior knowledge about electrical conductivity signal behavior in surgical conditions), the signal observed may be decomposed and interesting phenomenon including changepoints/changes in trend may be extracted.

Gaussian Process Change Point Detection is an extension of the BOCPD approach for detecting changepoints. See, e.g., Y. Saatçi et al., “Gaussian Process Change Point Models,” 927-934 (2010); see also R. Garnett et al., “Sequential Bayesian Prediction in the Presence of Changepoints and Faults,” The Computer Journal, vol. 53, no 9, p. 1430-1446, nov. 2010, doi: 10.1093/comjnl/bxq003. This probabilistic method allows use of Gaussian Processes to account for changepoints online, by combining them with a changepoint model designed to handle nonstationary time series data. Kernel based methods for detecting changepoints rely on a test statistic based upon the maximum kernel Fisher discriminant ratio as a measure of homogeneity between segments. See, e.g., Z. Harchaoui et al., “Kernel Change-point Analysis,” NIPS'08: Proceedings of the 21st International Conference on Neural Information Processing Systems, 609-616 (2008). Kernel changepoint analysis is a kernelized version of linear discriminant analysis, which maps observations onto a higher-dimensional feature space and detects changepoints by comparing the homogeneity of each subsequence, allowing performance of linear segmentation (either binary or non-binary). The goal of kernel changepoint analysis is to predict to which segment of data a data sample belongs, based on its value and other information. The main advantage of kernel changepoint analysis is to be non-parametric, e.g., based on either being distribution-free or having a specified distribution but with the distribution's parameters unspecified, although it relies heavily on the choice of the kernel function and its parameters. While kernel methods are usually used in supervised machine learning methods, the kernel changepoint analysis method offers an unsupervised method to detect trend changes/changepoints in signals.

Moreover, supervised machine learning may be used for online changepoint detection. Convolutional networks may be built using training data sets. See, e.g., S. Deldari et al., “Time Series Change Point Detection with Self-Supervised Contrastive Predictive Coding,” Proceedings of the Web Conference 2021, avr. 2021, p. 3124-3135. doi: 10.1145/3442381.3449903. Accordingly, with more extensive datasets available, the perforation detection algorithms described herein may be modified to implement machine learning methods that rely on supervised learning on a training dataset for online changepoint detection.

While various illustrative embodiments of the invention are described above, it will be apparent to one skilled in the art that various changes and modifications may be made therein without departing from the invention. The appended claims are intended to cover all such changes and modifications that fall within the true scope of the invention.

What is claimed:

1. A method implemented by a system comprising one or more processors for penetrating an anatomic structure, the method comprising:
causing a drilling portion to penetrate the anatomic structure;
receiving data indicative of electrical conductivity sensed by the drilling portion as the drilling portion penetrates the anatomic structure;
using a probabilistic perforation detection algorithm to probabilistically detect a breach condition based on the data; and
causing, if the breach condition is detected, the drilling portion to stop or modify penetration of the anatomic structure.

2. The method of claim 1, wherein using the probabilistic perforation detection algorithm to probabilistically detect the breach condition based on the data comprises using the Bayesian-based perforation detection algorithm to probabilistically detect a time instant when a probability distribution of a time series changes.

3. The method of claim 2, wherein using the Bayesian-based perforation detection algorithm to probabilistically detect the time instant when the probability distribution of the time series changes comprising using the Bayesian-based perforation detection algorithm to probabilistically detect the time instant when a run length associated with the data drops to zero.

4. The method of claim 1, wherein the probabilistic perforation detection algorithm comprises a Bayesian-based perforation detection algorithm, and wherein using the Bayesian-based perforation detection algorithm to probabilistically detect the breach condition based on the data comprises:
calculating a posterior predictive based on the data;
calculating a growth probability based on the posterior predictive;
calculating a changepoint probability;
calculating a marginal probability;
calculating a run length distribution based on the changepoint probability and the marginal probability;
computing a run length based on the run length distribution;
determining whether the run length exceeds a detection threshold once; and
probabilistically detecting the breach condition if the run length falls below the detection threshold after exceeding the detection threshold once.

5. The method of claim 4, using the Bayesian-based perforation detection algorithm to probabilistically detect the breach condition based on the data comprises assuming a changepoint prior is constant.

6. The method of claim 4, using the Bayesian-based perforation detection algorithm to probabilistically detect the breach condition based on the data comprises assuming a normal likelihood with an unknown mean and variance for the data.

7. The method of claim 6, wherein calculating the posterior predictive based on the data comprises using a conjugate exponential model to allow sequentially updating one or more distribution parameters as the data is received.

8. The method of claim 7, wherein using the conjugate exponential model comprises using a conjugate prior such that a posterior is in a same distribution as a prior.

9. The method of claim 8, wherein the conjugate prior is a Normal-Inverse-Gamma distribution.

10. The method of claim 8, wherein the run length distribution is a generalized Student's T distribution based on one or more distribution parameters.

11. The method of claim 10, further comprising:
initializing the one or more distribution parameters with one or more pro-perforation distribution priors,
wherein calculating the posterior predictive comprises using one or more post-perforation distribution priors.

12. The method of claim 4, wherein probabilistically detecting the breach condition if the run length falls below the detection threshold after exceeding the detection threshold once comprises probabilistically detecting the breach condition if the run length falls below the detection threshold in a predetermined [0, N] range.

13. The method of claim 4, further comprising modeling growth of an existing run associated with the data by calculating the posterior predictive using one or more distribution parameters associated with the data received at a first time for each run length value before updating each run length parameter value based on the data received at a second time.

14. The method of claim 1, wherein the probabilistic perforation detection algorithm comprises a Bayesian-based perforation detection algorithm, and wherein using the Bayesian-based perforation detection algorithm to probabilistically detect the breach condition based on the data does not require filtering of the data or prior calibration.

15. The method of claim 1, further comprising:
determining at least one of an entry point into the anatomic portion or a trajectory along which the drilling portion penetrates the anatomic structure; and
causing a robot arm coupled to the drilling portion to position the drilling portion in alignment with the at least one of the entry point or the trajectory.

16. The method of claim 15, wherein the robot arm is coupled to the drilling portion via a power drill unit mounted on a distal end of the robot arm, the power drill unit configured to transmit rotary motion to the drilling portion.

17. The method of claim 1, wherein causing the drilling portion to penetrate the anatomic structure comprises causing a power drill unit coupled to the drilling portion to transmit rotary motion to the drilling portion.

18. A system for penetrating an anatomic structure, the system comprising:
a drilling portion configured to sense electrical conductivity as the drilling portion penetrates the anatomic structure; and
a controller operatively coupled to the drilling portion, the controller programmed to:
cause the drilling portion to penetrate the anatomic structure;
receive data indicative of electrical conductivity sensed by the drilling portion as the drilling portion penetrates the anatomic structure;
use a probabilistic perforation detection algorithm to probabilistically detect a breach condition based on the data; and
cause, if the breach condition is detected, the drilling portion to stop or modify penetration of the anatomic structure.

19. The system of claim 18, wherein the probabilistic perforation detection algorithm comprises a Bayesian-based perforation detection algorithm configured to:
calculate a posterior predictive based on the data;
calculate a growth probability based on the posterior predictive;
calculate a changepoint probability;
calculate a marginal probability;
calculate a run length distribution based on the changepoint probability and the marginal probability;
compute a run length based on the run length distribution;
determine whether the run length exceeds a detection threshold once; and
probabilistically detect the breach condition if the run length falls below the detection threshold after exceeding the detection threshold once.

20. The system of claim 18, further comprising:
a robot arm; and
a power drill unit mounted on the robot arm, the power drill unit operatively coupled to the controller and configured to transmit rotary motion to the drilling portion to penetrate the anatomic structure.
